(19) United States
(12) Patent Application Publication
(10) Pub. No.: US 2025/0281436 A1
(43) Pub. Date: Sep. 11, 2025

(54) LUBRICANT

(71) Applicant: RECKITT BENCKISER HEALTH LIMITED, Berkshire (GB)

(72) Inventors: Philip TOWELL, Hull (GB); Natthira DANG-UTAI, Bangsamak, Bangpakong Chachoengsao (TH)

(21) Appl. No.: 18/261,290

(22) PCT Filed: Jan. 18, 2022
(86) PCT No.: PCT/GB2022/050134

§ 371 Date: Jul. 13, 2023

(30) Foreign Application Priority Data
Jan. 18, 2021 (GB) 2100631.7
Nov. 15, 2021 (TH) 2101007077

(51) Int. Cl. A61K 31/192; A61F 6/04; A61K 47/10; A61P 15/00

(52) U.S. Cl. CPC A61K 31/192; A61F 6/04; A61K 47/10; A61P 15/00

(57) ABSTRACT

A lubricant composition comprising: benzoic acid or a salt thereof in an amount of not more than 0.25% by weight; one or more monomeric polyols in a total amount of not more than 15% by weight: the composition having a pH in the range of 3.5 to 4.5: the composition having an osmolality of not more than 1200 mOsm/kg, wherein the lubricant composition comprises less than 1% by weight propane-1, 3-diol. Said composition for use for providing lubrication to the skin or a mucous membrane of a subject, preferably during sexual activity. A device comprising said lubricant composition. A product or kit comprising a condom and said lubricant composition packaged separately from the condom.

### FIELD OF THE INVENTION

The present invention relates to lubricants, and in particular to personal lubricants that are used to improve lubrication, moistening and comfort during sexual activity. The lubricant compositions provided herein provide moisture and lubricity whilst respecting the natural genital microbiota. Also provided are methods of using the personal lubricant compositions.

### BACKGROUND OF THE INVENTION

Personal lubricants are specialised lubricants which serve to reduce friction with body tissues during sexual activity. In particular, personal lubricants may be used to provide lubrication, or slip, during sexual activity by application directly to the vagina. For example, personal lubricants can be used to increase pleasure or reduce pain during sexual intercourse and can aid in reducing vaginal dryness. In medicine, personal lubricants may be employed for gynaecological examinations and the like.

Condoms may be pre-provided with a lubricant on their surface, but the technical considerations for such lubricants differ since the composition must be stable with the condom material (and the condom packaging material) for long storage periods. This invention primarily relates to personal lubricants which may be packaged separately from condoms, although the consumer may still choose to apply the lubricant to a condom just prior to sexual activity.

A wide variety of personal lubricants are currently available. They can be water-based or water-free, depending on the intended area of application and the required condom compatibility. Water-based lubricants generally function by supplying water on a body surface in a gelled or viscous form, by comprising a water-soluble polymer, such as a water-soluble cellulose derivative, or other water soluble polymers such as polyvinylpyrrolidone, polyvinyl alcohol and the like. In use on a body surface, these gelled or viscous systems retain water on the body surface to which they are applied, and the water provides lubrication. One or more humectants may be added to aid water retention on the body surface, so increasing the level of lubrication (that is, the lubricity of the formulation) provided, and/or increasing the length of time for which lubrication persists (that is, the longevity of lubricity).

Historically personal lubricants have tended to have an osmolality much higher than the typical vaginal osmolality of 200-300 mOsm/kg. It is now appreciated that the osmolality of a lubricant is important because the epithelial cells lining the vagina wall will constantly try and maintain homeostasis. Hyperosmolar lubricants cause the cells of the vagina to release fluid to dilute the lubricant. This may result in epithelial cell damage, which in turn results in an increased risk of infection due to the compromised epithelial barrier. There may also be increased dryness of the vagina. This is particularly problematic for menopausal females as the use of the hyperosmolar lubricant exacerbates vaginal dryness. In 2012, the World Health Organisation (WHO) recommended that healthcare organisations procure personal lubricants which have an osmolality of less than 1200 mOsm/kg. However, formulating a personal lubricant within this range is not straightforward whilst also balancing the need to achieve a number of other parameters including the required viscosity, slip properties, and rheological stability during storage.

Maintaining the PH balance of the vagina is important for preventing infection. A typical vaginal pH is between 3.8 and 4.5. A pH within or close to this range can help to keep bacterial and fungal infections at bay. On the other hand, the inclusion of certain ingredients to achieve the desired physical and other properties may drive the pH outside this range.

Microbiocides are needed especially within water-based personal lubricants to prevent microbial growth during storage. Unfortunately, questions have been raised over some microbiocides used in the past (e.g. polyquaternium-15) potentially causing epithelial damage, inflammation of the genital mucosa, alterations in the vaginal microbiome, and increased susceptibility to sexually transmitted infections.

A healthy vagina is dominated by Lactobacillus, which is a non-sporing gram-positive bacilli. Lactobacillus jensenii and Lactobacillus crispatus are the most numerous. The capacity of Lactobacilli bacteria to adhere and compete for adhesion sites in the vaginal epithelium and the capacity to produce antimicrobial compounds (hydrogen peroxide, lactic acid, bacteriocin-like substances), are important in the impairment of colonization by pathogens.

Clinical studies have demonstrated an association between the presence of hydrogen peroxide-producing strains of Lactobacillus and a decreased prevalence of gonorrhea, bacterial vaginosis (BV), and human immunodeficiency virus (HIV) infection. Bacterial vaginosis (BV) is characterised by an overgrowth of predominantly anaerobic organisms (such as Gardnerella vaginalis, Prevotella species, Mycoplasma hominis, and Mobiluncus species) and a loss of Lactobacilli. Some existing lubricants show a good log reduction of harmful bacteria such as S. aureus and C. albicans, but their formulations are too broad spectrum in their antimicrobial effect, such that desirable Lactobacilli levels also suffer as a result.

Visual stimulation is an important aspect of sexual activity, and it is therefore desirable that personal lubricants are aesthetically-pleasing, and in particular that they are transparent and uncoloured. The perception of touch is also an important aspect of sexual activity.

We have now recognised that there is a need for a personal lubricant that not only provides good lubrication and demonstrates antimicrobial effects against pathogens but also respects the body's pH levels, salt balance and natural microbiota and does not detract from sensual stimulation (e.g. visual and tactile stimuli). We also recognised it would be preferred for the lubricant to exhibit the rheological behavior of low shear viscosity for handling and application, but high shear viscosity and elasticity for in-use lubrication.

### SUMMARY OF THE INVENTION

According to a first aspect of the invention there is provided a lubricant composition comprising:
benzoic acid or a salt thereof in an amount of not more than about 0.25% by weight;
one or more monomeric polyols in a total amount of not more than about 15% by weight;
the composition having a pH in the range of about 3.5 to about 4.5;
the composition having an osmolality of not more than about 1200 mOsm/kg, and
wherein the lubricant composition comprises less than 1% propane-1,3-diol.

Lubricant compositions of the invention can provide not only an antimicrobial effect on pathogens that is comparable to some existing lubricants which contain parabens and chlorohexidine, but at the same time the lubricant respects the commensal Lactobaccilli, whilst matching the vagina's pH and osmolality. This is advantageous because the normal vaginal flora, acidic vaginal pH and vagina mucosa are all components of the innate defence mechanisms that protect against vulvovaginal infections.

The lubricant may be used as a personal lubricant.

In preferred embodiments, the lubricant is a gel. The lubricant may be used during sexual activity, or during medical examinations, such as gynecological and obstetric examinations.

Preferably the compositions of the invention are aqueous. Aqueous compositions are particularly preferred because they are compatible with most condom types, including natural rubber latex condoms, and can be easily washed-off. Accordingly, in an embodiment there is provided a lubricant composition comprising water as the carrier, solvent or base.

In some embodiments, the pH is between about 3.8 and about 4.2. In some embodiments, the pH is about 4.

All reference herein to the “% by weight” refers to “% by weight of the total composition”.

The lubricant composition includes benzoic acid or a salt thereof, such as sodium benzoate, as a preservative to prevent microbial growth.

The composition comprises not more than about 0.20% by weight of benzoic acid or a salt thereof. In some embodiments, the composition comprises between about 0.1% and about 0.2% by weight of benzoic acid or a salt thereof. In some embodiments, the composition comprises at least about 0.01%, or about 0.025%, or about 0.050%, or about 0.075%, or about 0.1%, or about 0.125%, or about 0.050%, or about 0.075%, by weight of benzoic acid or a salt thereof.

In some embodiments, the only preservative included within the lubricant composition is benzoic acid or a salt thereof.

Alternatively, the composition may comprise additional preservatives. An example of a suitable preservative is potassium sorbate.

A polyol is included within the composition as an emollient/humectant.

The composition comprises one or more monomeric polyols in a total amount of not more than about 15% by weight. In some embodiments, the composition may comprise a total amount of at least about 1% w/w, at least about 1.5% w/w, at least about 2% w/w, at least about 2.5% w/w, at least about 3% w/w, at least about 3.5% w/w, or at least about 4% w/w, of monomeric polyols. In some embodiments, the composition may comprise no more than about 4% w/w, no more than about 4.5% w/w, no more than about 5% w/w, no more than about 5.5% w/w, no more than about 6% w/w, no more than about 6.5% w/w, no more than about 7% w/w, no more than about 7.5% w/w, no more than about 8% w/w, no more than about 8.5% w/w, no more than about 9% w/w, no more than about 9.5% w/w, no more than about 10% w/w, no more than about 10.5% w/w, no more than about 11% w/w, no more than about 11.5% w/w, no more than about 12% w/w, no more than about 12.5% w/w, no more than about 13% w/w, no more than about 13.5% w/w, no more than about 14% w/w, no more than about 14.5% w/w, of the total amount of monomeric polyols.

In some embodiments, the composition comprises between about 4% and about 15% w/w of a total amount of monomeric polyol, preferably between about 5% and about 15%. In some embodiments, the composition comprises between about 5% and about 10% w/w total amount of monomeric polyol.

In some embodiments, the composition comprises about 5% w/w, or about 6% w/w, or about 7% w/w, or about 8% w/w, or about 9% w/w or about 10% w/w total amount of monomeric polyol.

In some embodiments, the one or more monomeric polyols may comprise or consist of one or more glycols, preferably a glycol. The composition may comprise between about 3% and about 15%, more preferably between about 5% and about 15%, between about 5% and about 10%, or between about 3% and about 8% by weight in total of one or more glycols.

In some embodiments the composition comprises glycol in no more than about 3% w/w, or no more than about 3.5% w/w, no more than about 4% w/w, no more than about 4.5% w/w, no more than about 5% w/w, no more than about 5.5% w/w, no more than about 6% w/w, no more than about 6.5% w/w, no more than about 7% w/w, no more than about 7.5% w/w, no more than about 8%, no more than about 8.5%, no more than about 9% w/w, no more than about 9% w/w, no more than about 9.5% w/w, no more than about 10% w/w, no more than about 10.5% w/w, no more than about 11% w/w, no more than about 11.5% w/w, no more than about 12% w/w, no more than about 12.5% w/w, no more than about 13% w/w, no more than about 13.5% w/w, no more than about 14% w/w, or no more than about 14.5% w/w, glycol.

The composition may comprise about 3.0% w/w, or about 3.5% w/w, or about 4.0% w/w, or about 4.5% w/w, or about 5.0% w/w, or about 5.5% w/w, or about 6.0% w/w, or about 6.5% w/w, or about 7.0% w/w, or about 7.5% w/w of a glycol.

The glycol component of the lubricant may comprise or consist of propane-1,2-diol (propylene glycol). Warming lubricants conventionally contain a high level of propane-1,2-diol.

In a particularly preferred embodiments of the lubricant composition, the only glycol present is propane-1,2-diol at a concentration of about 5% w/w, or about 6% w/w, or about 7% w/w or about 8% w/w.

In some other embodiments the composition comprises up to about 8% by weight of propane-1,2-diol in combination with a second monomeric polyol.

The second monomeric polyol may be a diol or a triol. An example of a suitable triol is glycerol.

The composition may comprise about 3% w/w, or about 3.5% w/w, or about 4% w/w, or about 4.5% w/w, or about 5% w/w, or about 5.5% w/w, or about 6% w/w, or about 6.5% w/w, or about 7% w/w, or about 7.5% w/w of glycerol.

In some embodiments, the lubricant composition comprises a combination of a monomeric polyol and a polymeric polyol. An example of a suitable polymeric polyol is a polymer of a glycol, such as polyethylene glycol.

The lubricant composition may comprise no more than 30% of a polyethylene glycol. This is one way to provide that the osmolality remains within acceptable WHO recommendation of less than 1200 mOsm/kg. Accordingly, the lubricant composition may comprise about 10% w/w, or about 15% w/w, or about 20% w/w, or about 25%/w or about 30% w/w of a polyethylene glycol.

Suitable, non-limiting examples of polyethylene glycols for use in the lubricant composition include PEG 400, PEG 500 and PEG 600. For example, the lubricant composition may comprise no more than about 30% PEG 400.

In some embodiments, the lubricant composition may comprise a combination of propane-1,2-diol and polyethylene glycol.

In a preferred embodiment, a mixture of propane-1,2-diol and glycerol provides a monomeric polyol component not more than about 8% w/w of the total composition. In a particularly preferred embodiment of the lubricant composition, propane-1,2-diol is present at about 3% w/w and glycerol is present at about 5% w/w.

In another preferred embodiment of the lubricant composition, propane-1,2-diol is present at about 4% w/w and polyethylene glycol is present at about 10% w/w. In an even more preferred embodiment of the lubricant composition, propane-1,2-diol is present at about 4% w/w and PEG 400 is present at about 10% w/w.

If a mixture of glycols is used, then the total glycol content preferably does not exceed about 15% w/w of the total weight of the composition, more preferably about 10% w/w of the total weight of the composition, and more preferably still about 8% w/w of the total weight of the composition.

A correlation has been identified between the molecular weight of glycols and osmolality values of the lubricant. Advantageously therefore, one preferred way to provide that the osmolality of the lubricant does not exceed the WHO recommendation of 1200 mOsm/kg, is to provide that the glycol used has a molar mass of no more than 650 g/mol. Preferably therefore, the glycol may have a molar mass of no more than 600 g/ml, or no more than 550 g/mol, or no more than 500 g/mol, or no more than 450 g/mol, or no more than 400 g/mol, or no more than 350 g/mol, or no more than 300 g/ml, or no more than 250 g/mol, or no more than 200 g/mol, or no more than 150 g/mol, or no more than 150 g/mol, or no more than 100 g/mol.

It is preferable that personal lubricants provide good levels of lubricity, and that the lubricity is long-lasting, to avoid the need for replenishing or “topping up” the amount of lubricant during use. The viscosity of a lubricant has an impact on the organoleptic characteristics of a product. The “skin feel” of a lubricant is important to consumers. For example, if the lubricant is perceived to be too thin and watery the user may doubt its lubricity properties. If the lubricant is perceived as being “sticky” rather than “silky” the user may consider that it will be difficult to rinse off. It may also be perceived as a cheaper alternative to a luxury product. Furthermore, in circumstances in which the lubricant is to be used during medical examinations, such as ultrasound, the user must be confident that it will provide the required cushioning effect and hydrodynamic lubrication. In general, personal lubricants are ideally non-tacky and do not string during use.

It is also preferable that personal lubricants do not interfere with the pleasurable sensual stimulation (e.g. visual, tactile, olfactory).

The rheological behaviours preferred by the inventors for this invention are a high viscosity at low shear rates for ease of handling and application and low viscosity at high shear rates for in-use lubrication. A lubricant with shear thinning characteristics will have a tendency to decrease in viscosity when driven to flow at high shear rates during sexual activity. This will provide lubricity between tissues thereby reducing friction during sexual intercourse whilst maintaining the sexual pleasure as users are able to feel the surfaces without pain.

The selection of both the humectant component and the viscosity-modifying polymer (when used) may influence the rheological properties of the lubricant.

Lubricant compositions that include propane-1,3-diol (propanediol) are often perceived by the consumer as having a light, water-like texture, with undesirable skin feel. Accordingly, the lubricant compositions of the present invention contain less than 1% propane-1,3-diol, preferably less than 0.5%, less than 0.1%, less than 0.05%, less than 0.01%, or substantially no propane-1,3-diol, preferably they are completely free from propane-1,3-diol. The inventors have developed lubricants with at least a comparable viscosity to a propane-1,3-diol-based lubricant but which have an improved skin feel.

Polyquaterium-15 (ethanaminium-N, N, N-trimethyl-2-[(2-methyl-I-oxo-2-propenyl)oxy]chloride polymer with 2-propenamide) is a formaldehyde-releasing preservative. The inclusion of this ingredient in cosmetic preparations may result in allergic contact dermatitis in some subjects. Accordingly, in some embodiments the lubricant composition may be free from Polyquaterium-15.

In some other embodiments, the lubricant composition is free from polyquaternium ammonium salts.

The composition may further include a pH adjuster. Non-limiting examples of suitable pH adjusters include organic acids and bases such as lactic acid, citric acid, triethanolamine and aminomethylpropanol and sodium hydroxide. The pH adjuster may be added in any suitable amount. Suitably, the pH adjuster is added in an amount suitable for adjusting the pH of the composition to the required pH.

The lubricant composition may further comprise a viscosity-modifying polymer. The viscosity-modifying polymer may be a thickening-polymer, also referred to as a “thickener”. Preferably, it is shear-thinning, preferably having a viscosity (when measured at a temperature of between 23° C. to 26° C. using a rotary viscometer) of at least 1000 cPs (preferably at least 1500 cPs, at least 2000 cPs, or at least 2500 cPs; and/or less than 6000 cPs, less than 5000 cPs, less than 4500 cPs, or less than 4000 cPs) at 1% concentration in water at a shear rate of 4 rpm. Preferably, it has a viscosity of at least 200 cPs (preferably at least 300 cPs, or at least 600 cPs; and/or less than 4000 cPs, less than 3000 cPs, or less than 2500 cPs) at 1% concentration in water at a shear rate of 20 rpm.

The viscosity-modifying polymer may be selected from a cellulosic polymer (e.g. microcrystalline cellulose, sodium carboxylmethyl cellulose), a carbomer, a xanthan gum, an alginate, an acrylate, a methacrylate, a silicone, a ceramide and a polyvinyl pyrrolidone, agar, locust bean gum and gum arabic. These are non-limiting examples of suitable viscosity-modifying polymers.

In some embodiments the lubricant composition includes a cellulosic polymer. The cellulosic polymer may be a hydroxyethylcellulose.

In an embodiment the lubricant composition comprises at least about 0.50% w/w of a hydroxyethylcellulose. The lubricant composition may comprise at least about 0.55% w/w, or at least about 0.60% w/w, or at least about 0.65% w/w, or at least about 0.70% w/w, or at least about 0.75% w/w, or at least about 0.80% w/w, or at least about 0.85% w/w, or at least about 0.90% w/w, or at least about 0.95% w/w, or at least about 1.00% w/w, or at least about 1.05% w/w, or at least about 1.10% w/w, or at least about 1.15% w/w, or at least about 1.20% w/w, or at least about 1.25% w/w, or at least about 1.30% w/w, or at least about 1.35% w/w, or at least about 1.40% w/w, or at least about 1.45% w/w, or at least about 1.50% w/w, or at least about 1.55% w/w, or at least about 1.60% w/w, or at least about 1.65% w/w, or at least about 1.70% w/w, or at least about 1.75% w/w, or at least about 1.80% w/w, or at least about 1.85% w/w, or at least about 1.90% w/w, or at least about 1.95% w/w, or at least about 2.00% w/w of a hydroxyethylcellulose.

In an embodiment the lubricant composition may comprise no more than about 2.00% w/w of a hydroxyethylcellulose. The lubricant composition may comprise no more than about 1.95% w/w, or about 1.90% w/w, or about 1.85% w/w, or about 1.80% w/w, or about 1.75% w/w, or about 1.70% w/w, or about 1.65% w/w, or about 1.60% w/w, or about 1.55% w/w, or about 1.50% w/w, or about 1.45% w/w, or about 1.40% w/w, or about 1.35% w/w, or about 1.30% w/w, or about 1.25% w/w, or about 1.20% w/w, or about 1.15% w/w, or about 1.10% w/w, or about 1.05%, w/w or about 1.00% w/w, or about 0.95% w/w, or about 0.90% w/w, or about 0.85% w/w, or about 0.80% w/w, or about 0.75% w/w, or about 0.70% w/w of a hydroxyethylcellulose.

The lubricant composition may comprise between about 0.50% w/w and about 2.00% w/w of a hydroxyethylcellulose.

The lubricant composition may comprise between about 0.50% w/w and about 1.00% w/w of a hydroxyethylcellulose.

The hydroxyethylcellulose preferably has a weight average molecular weight of 90,000 to 1,100,000 Da, preferably around 1,000,000 Da.

The hydroxyethylcellulose may be included as the only thickener within the lubricant composition. Alternatively, the hydroxyethylcellulose may be provided in combination with a second thickener.

In some embodiments the lubricant composition includes a carbomer as a thickener.

The lubricant composition may comprise at least about 0.10% w/w of a carbomer. The lubricant composition may comprise at least about 0.15% w/w, or at least about 0.20% w/w, or at least about 0.25% w/w, or at least about 0.30% w/w, or at least about 0.35% w/w, or at least about 0.40% w/w, or at least about 0.45% w/w, or at least about 0.50% w/w, or at least about 0.55% w/w, or at least about 0.60% w/w, or at least about 0.65% w/w, or at least about 0.70% w/w, or at least about 0.75% w/w, or at least about 0.80% w/w, or at least about 0.85% w/w, or at least about 0.90% w/w, or at least about 0.95% w/w, or at least about 1.00% w/w of a carbomer.

The lubricant composition may comprise no more that about 1.00% w/w of a carbomer. The lubricant composition may comprise no more than about 0.95% w/w, or about 0.90% w/w, or about 0.85% w/w, or about 0.80% w/w, or about 0.75% w/w. or about 0.70% w/w, or about 0.65% w/w, or about 0.60% w/w, or about 0.55% w/w, or about 0.50% w/w, or about 0.45% w/w, or about 0.40% of a carbomer.

The lubricant composition may comprise between about 0.25% w/w and about 1.00% w/w of a carbomer.

The lubricant composition may comprise between about 0.50% w/w and about 1.00% w/w of a carbomer.

The lubricant composition may comprise between about 0.25% w/w and about 0.50% w/w of a carbomer.

The carbomer is preferably a crosslinked homopolymer of acrylic acid, for example crosslinked with allyl sucrose or allyl pentaerythritol (an example of which is commercially available as carbomer 934P, 971 and 974P).

In some embodiments the lubricant composition includes xanthan gum as a thickener.

The lubricant composition may comprise at least about 0.10% w/w of a xanthan gum. The lubricant composition may comprise at least about 0.15% w/w, or at least about 0.20% w/w, or at least about 0.25% w/w, or at least about 0.25% w/w, or at least about 0.30% w/w, or at least about 0.35% w/w, or at least about 0.40% w/w, or at least about 0.45% w/w, or at least about 0.50% w/w, or at least about 0.55% w/w, or at least about 0.60% w/w, or at least about 0.65% w/w, or at least about 0.70% w/w, or at least about 0.75% w/w, or at least about 0.80% w/w, or at least about 0.85% w/w, or at least about 0.90% w/w, or at least about 0.95% w/w, or at least about 1.00% w/w, or at least about 1.05% w/w, or at least about 1.10% w/w, or at least about 1.15% w/w, or at least about 1.20% w/w, or at least about 1.25% w/w, or at least about 1.30% w/w, or at least about 1.35% w/w, or at least about 1.40% w/w, or at least about 1.45% w/w, or at least about 1.50% w/w of xanthan gum.

The lubricant composition may comprise no more that about 1.50% w/w of xanthan gum. The lubricant composition may comprise no more than about 1.45% w/w, or about 1.40% w/w, or about 1.35% w/w, or about 1.30% w/w, or about 1.35% w/w, or about 1.30% w/w, or about 1.25% w/w, or about 1.20% w/w of xanthan gum.

The lubricant composition may comprise between about 0.25% w/w and about 2.00% w/w of a xanthan gum.

The lubricant composition may comprise between about 0.50% w/w and about 1.50% w/w of a xanthan gum.

The lubricant composition may contain at least one of a hydroxyethylcellulose, a carbomer and a xanthan gum as a viscosity-modifying polymer. The composition may comprise a mixture of two or more thickeners.

The lubricant composition may contain a combination of a hydroxyethylcellulose and a carbomer.

The lubricant composition may contain a combination of a xanthan gum and a carbomer.

The lubricant composition may also contain polysaccharides such as carrageenans. These are linear sulphated polysaccharides that are extracted from red algae or seaweeds and which have gelling, thickening and stabilizing properties.

In some embodiments, the lubricant composition is substantially free of propylene glycol alginate (PGA). This is an ester of alginic acid in which some of the carboxyl groups are esterified with propylene glycol. More particularly the lubricant composition is substantially free from a propylene glycol alginate having a high degree of esterification, for example 82-85%. Such an alginate ester was found to have low viscosity Newtonian behaviour, like water, in contrast to other shear-thinning polymers like hydroxyethylcellulose and xanthan gum.

In some embodiments, the lubricant composition is substantially free of alginate.

In some embodiments, the lubricant composition is substantially free of alginic acid or algin.

The lubricant may further comprise an additional constituent selected from among flavouring agents, fragrances, aromas, warming agents, coolants, spermicides and colourants. Preferably, however, the lubricant is free of spermicide.

The lubricant composition may be made by a method comprising mixing together the components of the composition as generally described herein.

An example of the method of manufacture for the lubricant composition of Formula 1 (see Table 1) comprises the steps of:
1. Add purified water into main vessel;
2. Add propylene glycol into main vessel;
3. Charge benzoic acid (preservative) into main vessel.
4. Mix until homogenous.
5. Add purified water into main vessel;
6. Disperse thickeners into main vessel;
7. Mix until homogenous;
8. Adjust pH using sodium hydroxide to match with vaginal pH.

According to a second aspect of the invention there is provided the use of a lubricant composition of the first aspect of the invention for providing lubrication to the skin or a mucous membrane of a subject.

Preferably the use of the lubricant composition is during sexual activity.

According to a third aspect of the invention there is provided a method of lubricating the skin or a mucous membrane of a subject, comprising applying a lubricant composition of the first aspect of the invention to the skin or mucous membrane.

In some embodiments the method is a non-therapeutic method.

Preferably the lubricant composition is applied during before or during sexual activity.

According to a fourth aspect of the invention there is provided a device comprising the lubricant composition of the first aspect of the invention. The device may be a dispensing device. The device may be a pumped container.

According to a fifth aspect of the invention there is provided a product or kit comprising a condom and the lubricant composition of the first aspect of the invention packaged separately from the condom.

### BRIEF DESCRIPTION OF THE DRAWINGS

FIG. 1: Viscosities of gels with different humectants measured using a viscosity spindle at speed 20 rpm;

FIG. 2: Viscosities of gels with different humectants measured using a viscosity spindle at speed 50 rpm;

FIG. 3: Viscosity changes of gels of different humectants measured using a viscosity spindle at different speed (20 and 50 rpm) after 48 hours;

FIG. 4: H&E histology on Vaginal Epithelial Cells (VEC);

### DETAILED DESCRIPTION OF THE INVENTION

The following Examples illustrate embodiments of the invention.

### 1. Antimicrobial Properties

### 1.1. Formulations

The formulations of the exemplary lubricant compositions are shown in Table 1 below.

TABLE 1
FORMULATIONS

| | Comparative | Formula | Formula | Formula | Formula |
|---|---|---|---|---|---|
| | Lubricant | 1 | 2 | 3 | 4 |
| Chemical Name | % w/w | % w/w | % w/w | % w/w | % w/w |
| Purified water | 79.66 | 90.770 | 90.950 | 84.990 | 91.960 |
| Sodium hydroxide | 0.09 | 0.030 | 0.025 | 0.035 | 0.040 |
| Glycerine | 17 | | 5.000 | | |
| Propylene glycol | | 7.000 | 3.000 | 4.000 | 6.000 |
| Polyethylene glycol | | | | 10.000 | |
| Hydroxyethylcellulose | 2.30 | 1.800 | 0.750 | | |
| Xanthan gum | | | | 0.350 | 1.200 |
| Carbomer | | 0.200 | | 0.350 | 0.600 |
| Benzoic acid | | 0.200 | 0.200 | 0.200 | 0.200 |
| Methyl paraben | 0.20 | | | | |
| Chlorhexidine | 0.25 | | | | |
| Gluconate | | | | | |
| Glucono Delta Lactone | 0.50 | | | | |
| FCC | | | | | |
| Sodium saccharin | | | 0.075 | 0.075 | |
| TOTAL | 100.00 | 100.000 | 100.000 | 100.000 | 100.000 |
| pH | 4 | 4 | 4 | 4 | 4 |
| Viscosity (cPs) | 50,000-90,000 | 30,000-90,000 | 100-950 | 400-1,400 | 3500-7500 |
| Osmolality (mOsm/kg) | 2007 | 973 | 789-973 | 630 | 846-853 |

### 1.2. Methodology

The exemplary formulations were tested for antimicrobial activity based on the European Pharmacopoeia (Ph Eur) and United States Pharmacopoeia (USP) Preservative Efficacy Test. These methods are incorporated by reference herein.

The test organisms were:
Staphylococcus aureus NCTC 10788/ATCC 6538
Pseudomonas aeruginosa NCIMB 8626/ATCC 9027
Escherichia coli NCIMB 8545/ATCC 8739
Candida albicans NCPF 3179/ATCC 10231
Lactobacillus gasseri NCIMB 702820
Lactobacillus crispatus NCIMB 4504

Three replicates of each formulation were tested with 10 g of sample inoculated at one inoculum level of approx. 10⁶ cfu/g.

Three timepoints were assessed at 5, 30 and 120 minutes.

Enumeration of the number of surviving microorganisms was assessed by serial dilution and duplicate plate count.

### 1.3. Results

The results are shown in Table 2 below. A topical preparation passes the preservative tests if the following criteria are met:

TABLE 2
ANTIMICROBIAL EFFECT OF LUBRICANT COMPOSITIONS

| Test | Bacteria | Yeast and Molds |
|---|---|---|
| Ph Eur | Minimum Log 2 reduction in | Minimum Log 2 reduction |
| | 48 hours, Log 3 reduction in | in 14 days with no |
| | 7 days with no increase | increase thereafter |
| | thereafter | |
| USP | Not less than Log 2 reduction | No increase from the |
| | from the initial count at 14 | initial calculated |
| | days and no increase from 14 | count at 14 and 28 days |
| | days count to the 28 days | |
| | count | |

Table 2 (a)-(e) demonstrates the antimicrobial effects of the lubricant composition according to the invention as compared to Comparative Lubricant.

(a) Comparative Lubricant

| | Log reduction | | |
|---|---|---|---|
| Organism | 5 Minutes | 30 Minutes | 120 Minutes |
| S. aureus | 0.67 | 2.02 | 4.61 |
| P. aeruginosa | >5.68 | >5.68 | >5.68 |
| E. coli | >5.56 | >5.56 | >5.56 |
| C. albicans | 2.48 | 3.25 | 3.84 |
| L. gasseri | 2.96 | 3.73 | 4.14 |
| L. crispatus | >4.02 | >4.02 | >4.02 |

(b) Antimicrobial effect of Formula 1

| | Log reduction | | |
|---|---|---|---|
| Organism | 5 Minutes | 30 Minutes | 120 Minutes |
| S. aureus | 0.77 | 0.86 | >5.64 |
| P. aeruginosa | 1.87 | >5.90 | >5.90 |
| E. coli | 1.20 | 1.69 | 3.22 |
| C. albicans | 0.93 | 0.90 | 0.31 |
| L. gasseri | 1.51 | 0.54 | 0.62 |
| L. crispatus | 1.26 | 1.35 | 1.24 |

(c) Antimicrobial effect of Formula 2

| | Log reduction | | |
|---|---|---|---|
| Organism | 5 Minutes | 30 Minutes | 120 Minutes |
| S. aureus | 0.10 | −0.01 | 1.02 |
| P. aeruginosa | 0.47 | 4.29 | >5.95 |
| E. coli | 0.14 | 0.52 | 1.85 |
| C. albicans | 1.01 | 0.98 | No data |
| L. gasseri | 0.83 | 0.98 | 1.33 |
| L. crispatus | 0.48 | 0.6 | 1.22 |

(d) Antimicrobial effect of Formula 3

| | Log reduction | | |
|---|---|---|---|
| Organism | 5 Minutes | 30 Minutes | 120 Minutes |
| S. aureus | 0.04 | 0.00 | 0.22 |
| P. aeruginosa | 0.63 | 2.33 | >5.95 |
| E. coli | −0.01 | 0.25 | 0.68 |
| C. albicans | 1.03 | 1.09 | 1.01 |
| L. gasseri | 0.68 | 0.59 | 0.91 |
| L. crispatus | 0.59 | 0.48 | 0.75 |

(e) Antimicrobial effect of Formula 4

| | Log reduction | | |
|---|---|---|---|
| Organism | 5 Minutes | 30 Minutes | 120 Minutes |
| S. aureus | 0.20 | 0.18 | >5.68 |
| P. aeruginosa | 5.00 | >5.95 | >5.95 |
| E. coli | 0.62 | 1.01 | 2.84 |
| C. albicans | 2.12 | 1.63 | 1.02 |
| L. gasseri | 1.86 | 1.32 | 1.00 |
| L. crispatus | 0.99 | 0.83 | 0.71 |

The results demonstrate that at 120 minutes, the compositions of Formulas 1 and 4 have a comparable Log reduction against S. aureus and P. aeruginosa as Comparative Formula 1. Notably, whilst Comparative Formula 1 has a Log reduction against the Lactobaccilli species of over 4, Formulas 1 and 4 respect this population of bacteria, with far lower Log reductions of around 1.

The results also demonstrate that at 120 minutes, the composition of Formulas 2 and 3 have a comparable Log reduction against P. aeruginosa as Comparative Formula 1, whilst also respecting the Lactobaccilli species of bacteria, again with Log reductions of around 1.

### 2. The Effect of Humectant Selection on Viscosity

### 2.1. Formulations

Four different types of humectants were used to investigate the impact of varying the humectant on the viscosity of the lubricant.

TABLE 3

| | Material | | Concentration (% w/w) | | | |
|---|---|---|---|---|---|---|
| No. | Name | Function | Example 1 | Example 2 | Example 3 | Example 4 |
| 1. | Xanthan gum | Thickener | 1.50 | 1.50 | 1.50 | 1.50 |
| 2. | Propylene | Humectant | 6.00 | — | — | — |
| | Glycol | | | | | |
| | (propane 1,2- | | | | | |
| | diol) | | | | | |
| 3. | Glycerol | | — | 6.00 | — | — |
| 4. | Propanediol | | — | — | 6.00 | — |
| | (propane-1,3- | | | | | |
| | diol) | | | | | |
| 5. | Polyethylene | | — | — | — | 6.00 |
| | Glycol | | | | | |
| 6. | Purified Water | Solvent | 92.50 | 92.50 | 92.50 | 92.50 |

### 2.2. Methodology

The viscosities were measured at Tₒ and after 2 days using a viscosity spindle at speeds of 20 rpm and 50 rpm. Data were expressed as mean±SD and the results were analysed. Following ANOVA, the means were separated using Tukey (p<0.05).

### 2.3. Results

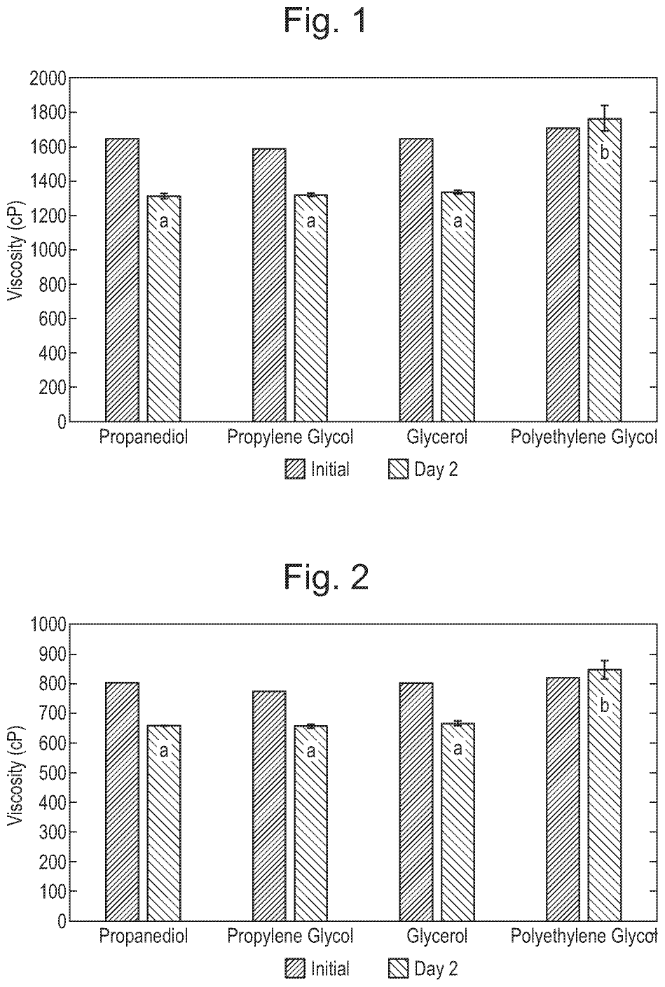

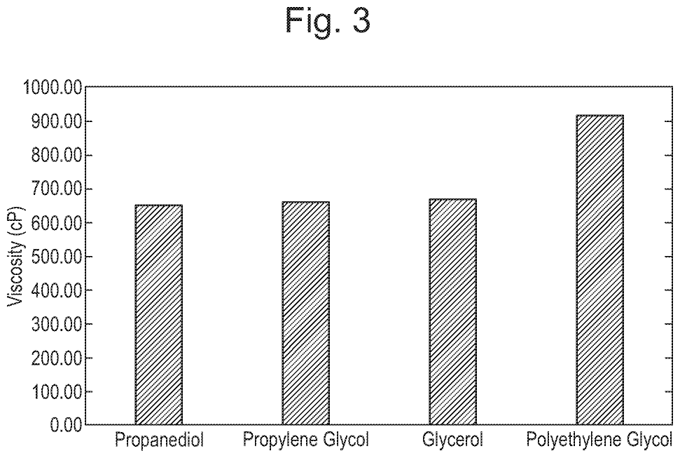

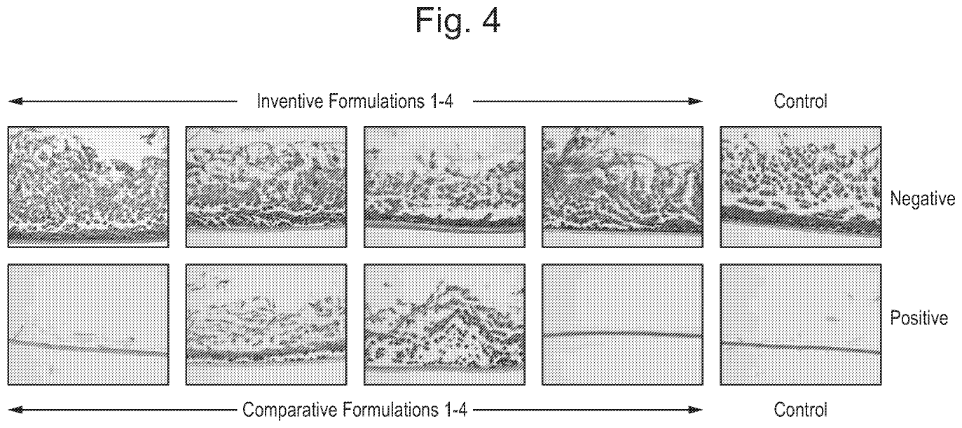

The viscosities of the lubricants measured at speed 20 rpm and 50 rpm are illustrated in FIGS. 1 and 2, respectively.

The Figures illustrate that the initial viscosities measured at 20 rpm ranged between 1590 to 1710 cPs with 15.9 to 17.2% torque while that measured with at 50 rpm ranged between 776 to 820 cPs with 19.4 to 10.5% torque.

After 2 days, the viscosities of formulations with propane-1,3-diol, propane-1,2-diol and glycerol decreased while that of polyethylene glycol increased. The viscosities of formulations with propane-1,3-diol, propane-1,2-diol and glycerol after 2 days showed 16.98 to 20.42% reduction when measured at low speed while those measured at high speed showed 15.34 to 17.91% reduction. In contrast, the viscosity of polyethylene glycol was increased 3.33% when measured at 20 rpm and 3.41% increased when measured at 50 rpm.

The viscosity of formulation that contained polyethylene glycol was significantly higher than those other humectants. Statistical analysis showed that there was no significant difference of viscosities of formulations that composed of propane-1,3-diol, propane-1,2-diol and glycerol.

In conclusion, lubricants that contain propane-1,2-diol and glycerol have a viscosity comparable to propane-1,3-diol, but have an improved skin feel, in that they do not feel “watery”. Lubricants that contain polyethylene glycol have an improved viscosity when compared to propane-1,3-diol.

### 3. Percent Cell Viability of Vaginal Epithelial Cells

### 3.1. Formulations

The exemplary inventive Formulations 1˜4 as outlined in Section 1.1, in addition to the Comparative Formula 1 were used. In addition, three other Commercially available lubricants (Comparative Formulas 2-4) were used.

### 3.2 Methodology

### Cell Culture

Vaginal Epithelial Cell (VEC) cultures (with a surface area 0.33 cm²) were equilibrated overnight after receipt in a humidified incubator at 37° C. and 5% CO₂.

Controls and test formulations (75 μL; ca 227 μl/cm²) were applied to the apical side of the tissues (n=1 or 3). Sterile culture media was included as the negative control and 1% Triton™ X-100 was included as the positive control. Samples were placed in the incubator for 24 hours.

### Cell Viability

Percent cell viability of VEC was calculated to the negative control (media). Tissue was placed into 1 mg/ml MTT substrate in culture media and incubated for 3 hours followed by extraction in isopropyl alcohol and quantified on a BioTek Synergy HT plate reader at OD 570 nm. Data is shown as the mean±standard deviation (n=1-3)

### Histology

Tissue was fixed overnight in 10% normal buffered formalin for histology followed by dehydration and paraffin embedding. The paraffin blocks were cut at ca. 5 μm and stained using hematoxylin and eosin (H&E). The H&E score was assessed by visual comparison based on the disruption of the basal and parabasal cell layers compared to negative control tissue.

Scale of 0-4, as follows:
0=indistinguishable from negative control
1=increased parabasal cell disruption
2=disorganization of basal cell layer
3=increased disorganization of basal and parabasal cell layer
4=most extensively disrupted, separation of epithelial layers from porous support.

### 3.3 Results

| Treatment | % Viability Mean | H&E score |
|---|---|---|
| Formula 1 (n = 3) | 124.32% | 1 |
| Formula 2 (n = 3) | 99.56% | 1 |
| Formula 3 (n = 3) | 74.76% | 1 |
| Formula 4 (n = 3) | 102.99% | 1 |
| Comparative Formula 1 (n = 1) | 29.69% | 3 |
| Comparative Formula 2 (n = 1) | 28.23% | 4 |
| Comparative Formula 3 (n = 1) | 5.56% | 4 |
| Comparative Formula 4 (n = 1) | 63.56% | 2 |
| Negative control (n = 3) | 100% | 0 |
| Positive control (n = 3) | 1.67% | 4 |

### MIT Cell Viability

Data is shown as the mean±standard deviation (n=1-3).

All inventive formulations (Formulas 1-4) performed comparably to the negative control (media) in measured % cell viability of between 75%-124%.

Comparative Formulas 1˜4 (Comparative Formulas 1-4) had a measured % cell viability of between 6%-64%.

### Histology

Histological assessment by H&E showed correlation between VEC integrity and MTT % viability, with all the inventive formulations (Formula 1-4) scoring comparably to the negative control.

Positive control treatment with 1% Triton-X resulted in complete removal of the cell layers from the porous support. Comparative Formulas 2 and 3 scored comparably to the positive control.

### CONCLUSION

All inventive formulations (Formulas 1-4) resulted in greater cell viability compared to all existing Commercially available formulations and had percent viabilities similar to that of negative control tissues.

Histological observation supported the trend of lack of irritation in the inventive formulations compared to Existing formulations with greater retention of basal and parabasal cell layer integrity.

1. A lubricant composition comprising:
benzoic acid or a salt thereof in an amount of not more than about 0.25% by weight;
one or more monomeric polyols in a total amount of not more than about 15% by weight; and
propane-1,3-diol in an amount of less than 1% by weight,
wherein the lubricant composition has a pH in the range of about 3.5 to about 4.5, and
wherein the lubricant composition has an osmolality of not more than about 1200 mOsm/kg.

2. The lubricant composition of claim 1, wherein at least one of the one or more monomeric polyols is a glycol.

3. The lubricant composition of claim 2, wherein the composition comprises between about 3% and about 15% by weight of the glycol.

4. The lubricant composition of claim 2, wherein the glycol is propane-1,2-diol.

5. The lubricant composition of claim 4, wherein the composition comprises between about 3% and about 8% by weight of propane-1,2-diol in combination with a second monomeric polyol.

6. The lubricant composition of claim 5, wherein the second monomeric polyol is a diol or a triol.

7. The lubricant composition of claim 1, wherein the lubricant composition further includes a polymeric polyol, preferably polyethylene glycol.

8. The lubricant composition of claim 7, wherein the lubricant composition comprises up to about 10% by weight of polyethylene glycol.

9. The lubricant composition of claim 1, wherein the lubricant composition is free from polyquaternium-15.

10. The lubricant composition of claim 9, wherein the lubricant composition is free from quaternary ammonium salts.

11. The lubricant composition of claim 1, further comprising a viscosity-modifying polymer.

12. The lubricant composition of claim 1, wherein the composition is aqueous.

13. A method of using a lubricant composition according to claim 1 for providing lubrication to skin or a mucous membrane of a subject, preferably during sexual activity, the method comprising applying the lubricant composition to the skin or mucous membrane.

14. A method of lubricating skin or a mucous membrane of a subject, comprising applying a lubricant composition according to claim 1 to the skin or mucous membrane, preferably wherein the method is a non-therapeutic method.

15. The method of claim 14, wherein the lubricating of the skin or mucous membrane of the subject is during sexual activity.

16. A device comprising the lubricant composition of claim 1, preferably wherein the device is a container with a pump dispenser.

17. A product or kit comprising a condom and the lubricant composition of claim 1 packaged separately from the condom.

18. The lubricant composition of claim 2, wherein the lubricant composition comprises between about 3% and about 8% by weight of the glycol.

19. The lubricant composition of claim 5, wherein the second monomeric polyol is a glycerol.

20. The lubricant composition of claim 11, wherein the viscosity-modifying polymer is:
a) selected from the group consisting of a cellulosic polymer, a carbomer, and a xanthan gum; or
b) not a propylene glycol alginate.
